(19) United States
(12) Patent Application Publication
(10) Pub. No.: US 2009/0017078 A1
(43) Pub. Date: Jan. 15, 2009

(54) Anhydrous topical formulation for polyphenols

(75) Inventors: Matthew Buderer, Oak Harbor, OH (US); Peter Ford, New Brunswick (CA); George Roentsch, Stoddard, NH (US); Robert Cervelli, Nova Scotia (CA); Heather Joyce, Nova Scotia (CA)

Correspondence Address: DLA PIPER LLP (US), 4365 EXECUTIVE DRIVE, SUITE 1100, SAN DIEGO, CA, 92121-2133, US

(73) Assignee: Origin Biomedicinals, Inc., Halifax, NS (CA)

(21) Appl. No.: 10/570,481

(22) PCT Filed: Sep. 21, 2004
(86) PCT No.: PCT/CA2004/001659

§ 371 Date: Aug. 25, 2008

Related U.S. Application Data
(60) Provisional application No. 60/504,972, filed on Sep. 23, 2003.

(51) Int. Cl. A61K 9/06; A61K 31/05

(52) U.S. Cl. 424/400; 514/731

(57) ABSTRACT

The present invention discloses a composition of matter, and method to formulate same, which is an anhydrous topical cream, gel or ointment base, a polyphenol and a suitable adsorbent binding carrier to which the polyphenol will bind for purposes of even disbursement within the cream, gel or base and which will not inhibit the ability of the polyphenols to be released on and into the aqueous environment of the skin when the topical mixture is applied thereto. The binding carrier provides the ability to disperse a hydrophilic polyphenol in a non-aqueous medium for purposes of topical application to the body. In particular, the present invention discloses the use of polyphenols such as tea catechins, and in particular green tea catechins, disbursed in an anhydrous base consisting of either saturated or unsaturated plant oils or waxes through the use of a variety of binding carriers including, but is not limited to, talcs and clays, alginates, algae, agars, gums, gelatins, celluloses, silica, silica gels, simethicone, salicylates, silicates and silicone resins, tragacanths, calcium carbonates and magnesium or zinc oxides. Such binding carriers are particularly useful when polyphenol concentrations exceed 0.2% w/w in the mixture, and their use is preferred when concentrations are between 1.0 to 20% w/w polyphenols.

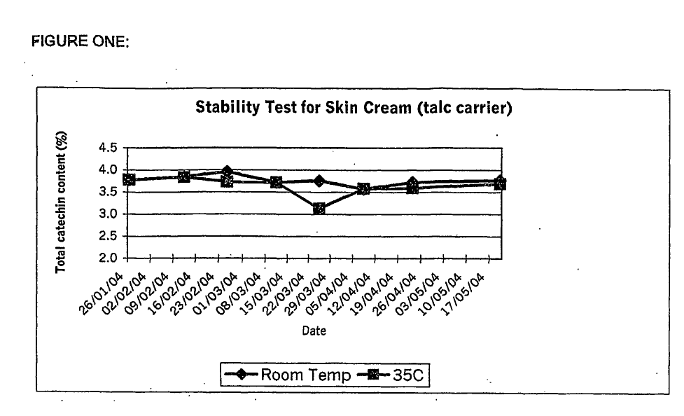

### BACKGROUND TO THE INVENTION

Plant polyphenols are known to be potent anti-oxidants and are recognized as important components of dietary health. Increasingly, polyphenols derived from tea, grape and other plant sources are purified and can be taken as dietary supplements for additional beneficial effect. It is becoming recognized that polyphenols can be applied topically to the skin and will confer the same beneficial effects locally to the skin and surrounding tissues.

Many polyphenols, especially the green tea catechins, however, are very unstable at room temperature and are oxidized and break down within days, especially in the presence of water (1). In order to assure the stability of polyphenols in topical mixtures, it is possible to formulate such mixtures without water (anhydrous), and to thereby increase the stability of the polyphenols. Other antioxidants such as vitamin C will add to the stabilizing effect. Plant oils, either saturated or unsaturated, are commonly used as a base in a variety of commercial topical mixtures, but polyphenols are poorly soluble in these oils. Many polyphenols, especially the green tea extracts, and more especially green tea extracts which have been enriched for polyphenols, do not solubilize or disperse evenly in anhydrous topical mixtures composed primarily of oils or waxes.

For this reason, it is important to devise a means by which polyphenols can be evenly disbursed in anhydrous topical mixtures in order to provide a suitable product that assures polyphenol stability while also providing a suitable commercial appeal to a topical product. Such even disbursement throughout an anhydrous topical mixture can be accomplished through the use of an inert absorbent binding carrier which will not inhibit the ability of the polyphenols to be released on and into the aqueous environment of the skin when the topical mixture is applied thereto.

The principles of adsorption are well recognized in the pharmaceutical compounding profession, and are used for decolorising solutions, adsorption chromatography, detergency and wetting. Drugs such as dues, alkaloids, fatty acids and inorganic acids and bases can be adsorbed onto solids such as charcoal and alumina. A novel application for the adsorption of polyphenols prior to use in anhydrous mixtures is described herein.

### REFERENCES CITED

1. Zhou, Q., et al. (2003) Investigating the stability of EGCg in Aqueous Media. Current Separations 20:3.
2. Proniuk, S., et al (2002) Preformulation study of epigallocatechin gallate, a promising antioxidant for topical skin cancer prevention. J Pharm Sci 2002 January; 91(1):111-6

### BRIEF SUMMARY OF THE INVENTION

The present invention discloses a composition of matter, and method to formulate same, which is an anhydrous topical cream, gel or ointment base, a polyphenol and a suitable adsorbent binding carrier to which the polyphenol will bind for purposes of even disbursement within the cream, gel or ointment base, and which will not inhibit the ability of the polyphenols to be released on and into the aqueous environment of the skin when the topical mixture is applied thereto. The binding carrier provides the ability to disperse a hydrophilic polyphenol in a non-aqueous medium for purposes of topical application to the body. In particular, the present invention discloses the use of polyphenols such as green tea catechins disbursed in an anhydrous base consisting of either saturated or unsaturated plant oils or waxes through the use of a variety of binding carriers, including by not limited to talc, clay or silica, salicylates, silicates and silicone resins, agars, alginates, gums, celluloses, tragacanths, calcium carbonates and magnesium or zinc oxides. Such binding carriers are particularly useful when polyphenol concentrations exceed 0.2% w/w in the topical mixture, and their use is preferred when concentrations are between 1.0 to 20% w/w polyphenols.

### DETAILED DESCRIPTION OF THE INVENTION

Plant polyphenols are known to be potent anti-oxidants and anti-tumor-agents and are recognized as important components of dietary health. Increasingly, polyphenols derived from tea, grape, olive and other plant sources are purified and can be taken as dietary supplements for additional beneficial effect. Examples include catechins, hydroxytyrosols and proanthocyanidins. Green tea, for example, contains a class of polyphenols called catechins. Catechins have been shown in a number of studies to confer benefits for weight loss, halitosis, numerous cancers, arthritis and allergies. It is becoming recognized that polyphenols can be applied topically to the skin and will confer the same beneficial effects locally to the skin and surrounding tissues. Studies of topical application of polyphenols have shown possible benefit for UV damage, pre-cancerous skin lesions and skin cancers, as well as being a general healing agent.

Many polyphenols, especially the green tea catechins, however, are very unstable at room temperature and are oxidized and break down within days, especially in the presence of water (1). In order to assure the stability of polyphenols in topical mixtures, it is possible to formulate such mixtures without water (anhydrous), utilizing various types of artificial or natural oils, waxes and emulsifiers, and to thereby increase the stability of the polyphenols. Other antioxidants or preservatives such as vitamin C or EDTA will add to the stabilizing effect. Plant oils or waxes, either saturated or unsaturated, are commonly used in a variety of commercial topical mixtures. Examples may include shea butter, aloe vera, almond oil, olive oil, avocado oil, coconut oil, jojoba oil, avena sativa oil and others. Polyphenols are generally hydrophilic and are thus poorly soluble in most oils or waxes that are commonly used for topical preparations. Some polyphenols are resin-like when is concentrated or purified form.

For this reason, it is important to devise a means by which polyphenols can be evenly disbursed in anhydrous topical mixtures, particularly artificial or natural oils and waxes, in order to provide a suitable product that assures polyphenol stability while also providing a suitable appearance, texture and appeal for a commercial topical product. Such even disbursement throughout an anhydrous topical mixture can be accomplished through the use of an adsorbent binding carrier which will not inhibit the ability of the polyphenols to be released on and into the aqueous environment of the skin when the topical mixture is applied thereto.

In the present invention, a number of means are disclosed by which a suitable binding carrier is added to an anhydrous topical mixture along with a polyphenol to achieve such a disbursement throughout the mixture. Such carriers are generally described as compounds or complex organic compounds which are generally regarded as safe for use on the skin and may also add benefit to the skin, possibly with a high melting point, which may or may not be absorbed into the skin but which release the drug or target compound being adsorbed to the carrier upon contact with the aqueous environment of the skin for absorption into the skin. Examples of such carriers include, but are not limited to, talcs and clays (such as attapulgite, halloysite and kaolin); alginates, algae, agars, gums, gelatins and celluloses; silica, silica gels, simethicone, salicylates, silicates and silicone resins (such as polymethylsilsequioxane); tragacanths; charcoal, calcium carbonates; and magnesium or zinc oxides.

An important method of combining an anhydrous mixture, polyphenol and binding carrier is to first triturate the polyphenol and binding carrier until uniform, providing an opportunity for the polyphenol to be adsorbed onto the binding carrier. Heat may be required during this process depending on the type and concentration of polyphenol and binding carrier. Friction techniques like grinding or milling may also facilitate the adsorption to a binding carrier. This polyphenol/carrier can then be added to the anhydrous mixture to achieve a uniformly disbursed topical mixture.

Such a composition and method of using a suitable binding carrier to evenly disperse polyphenols in an anhydrous topical mixture is not readily obvious to one skilled in the art. At least one reference was able to circumvent the problem of uneven solubilization and disbursement through the use of glycerin-based mixtures, which are not artificial or natural oils and waxes, and which do not have the commercial value for topical preparations (2). Said binding carriers as disclosed in the present invention are particularly useful when polyphenol concentrations exceed 0.2% w/w in the mixture, and their use is preferred when concentrations are between 1.0 to 20% w/w polyphenols.

### EXAMPLE ONE

### Acne Cream

### Using Salicylic Acid

An anhydrous preparation for the treatment of acne consisting of 3% w/w purified green tea extract (comprising at least 70% polyphenols) has been devised which utilizes 0.5% w/w salicylic acid as a suitable binding carrier as follows:
77.1% jojoba oil
15.0% bees wax
2.0% lecithin
2.0% ascorbyl palmitate (vitamin C)
0.2% sorbic acid
3.0% green tea polyphenol extract (70% polyphenols)
0.5% salicylic acid
0.2% tea tree oil

It is necessary during the formulation process to first triturate the polyphenol and binding carrier until uniform using gentle heat and milling. This polyphenol/carrier can then be added to the balance of the anhydrous mixture to achieve a uniformly disbursed topical mixture upon further mixing and/or milling.

### EXAMPLE TWO

### Skin Cream

### Using Silica Gel

A preparation for the treatment of damaged skin consisting of 5% w/w purified green tea extract (comprising at least 70% polyphenols) has been devised which utilizes 6% w/w micronized silica gel as a suitable binding carrier as follows:
67.5% jojoba oil
5.0% dimethyl sulfone
12.0% beeswax
2.0% lecithin
6.0% silica gel (micronized)
5.0% green tea polyphenol extract (70% polyphenols)
0.2% sorbic acid
2.0% ascorbyl palmitate (vitamin C)
0.2% Lavender oil
0.1% Tea tree oil

It is necessary during the formulation process to first triturate the polyphenol and binding carrier until uniform. This polyphenol/carrier can then be added to the balance of the anhydrous mixture to achieve a uniformly disbursed topical mixture upon further mixing and/or milling.

### EXAMPLE THREE

### Skin Cream

### Using Talc

A preparation for the treatment of damaged skin consisting of 5% w/w purified green tea extract (comprising at least 70% polyphenols) has been devised which utilizes 20% w/w talc as a suitable binding carrier as follows:
58.5% jojoba oil
12.0% beeswax
2.0% lecithin
20.0% talc
5.0% green tea polyphenol extract (70% polyphenols)
0.2% sorbic acid
2.0% ascorbyl palmitate (vitamin C)
0.2% Lavender oil
0.1% Tea tree oil

It is necessary during the formulation process to first triturate the polyphenol and binding carrier until uniform. This polyphenol/carrier can then be added to the balance of the anhydrous mixture to achieve a uniformly disbursed topical mixture upon further mixing and/or milling.

### EXAMPLE FOUR

### Skin Cream

### Using Kaolin Clay

A preparation for the treatment of damaged skin consisting of 5% w/w purified green tea extract (comprising at least 70% polyphenols) has been devised which utilizes 6% w/w micronized silica gel as a suitable binding carrier as follows:
74.5% jojoba oil
6.0% beeswax
2.0% lecithin
10.0% kaolin china clay
5.0% green tea polyphenol extract (70% polyphenols)
0.2% sorbic acid
2.0% ascorbyl palmitate (vitamin C)
0.2% Lavender oil
0.1% Tea tree oil

It is necessary during the formulation process to first triturate the polyphenol and binding carrier until uniform. This polyphenol/carrier can then be added to the balance of the anhydrous mixture to achieve a uniformly disbursed topical mixture upon further mixing and/or milling.

### EXAMPLE FIVE

### Wart Cream

### Using Silica Gel

A preparation for the treatment of warts consisting of 12% w/w purified green tea extract (comprising at least 70% polyphenols) has been devised which utilizes silica gel as a binding carrier as follows:
62.7.0% shea butter
5.0% avena sativa oil
10.0% beeswax
8.0% silica gel (micronized)
12.0% green tea polyphenol extract (70% polyphenols)
2.0% ascorbyl palmitate (vitamin C)
0.2% eucalyptus oil
0.1% tea tree oil

It is necessary during the formulation process to first triturate the polyphenol and binding carrier until uniform. This polyphenol/carrier can then be added to the balance of the anhydrous mixture to achieve a uniformly disbursed topical mixture upon further mixing and/or milling.

### EXAMPLE SIX

### Lip Balm

### Using Zinc Oxide

A preparation for the treatment of the lips consisting of 10% w/w purified green tea extract (comprising at least 70% polyphenols) has been devised which utilizes 1% w/w zinc oxide as a binding carrier as follows:
10% green tea polyphenol extract (70% polyphenols)
0.5% allantoin
0.2% prunella vulgaris extract
0.3% geranium oil
0.1% tea tree oil
0.1% bergamot oil
1.0% zinc oxide
0.1% vitamin C
3 i.u./gm vitamin E
0.5% peppermint oil flavoring
1% avena sativa oil
71.7% shea butter
15% bees wax

It is necessary during the formulation process to first triturate the polyphenol and binding carrier until uniform. This polyphenol/carrier can then be added to the balance of the anhydrous mixture to achieve a uniformly disbursed topical mixture upon further mixing and/or milling.

In order to test the stability of the polyphenol catechins in an anhydrous topical formulation prepared according to the present invention, the skin cream formulation which uses talc (Example Three) as an adsorbent binding carrier was chosen for stability testing of polyphenols. Catechin content was measured using standard chromatography techniques on said skin cream stored at room temperature and at 35° C. over a 5 month period. The results are indicated below.

TABLE ONE
Catechin content of skin cream (talc carrier) stored at room temp. and 35° C.

| | | | | | | | | | | |
|---|---|---|---|---|---|---|---|---|---|---|
| | Gallic | | | | | | | | | Catechin |
| Date | Acid | EGC | GC | EC | C | EGCg | GCg | ECg | Cg | Total |

ROOM TEMPERATURE

| | | | | | | | | | | |
|---|---|---|---|---|---|---|---|---|---|---|
| 04 Jan 26 | 0.0 | 0.9 | 0.3 | 0.4 | 0.1 | 1.5 | 0.1 | 0.5 | 0.0 | 3.8 |
| 04 Feb 12 | 0.0 | 0.9 | 0.3 | 0.4 | 0.1 | 1.6 | 0.2 | 0.5 | 0.0 | 3.8 |
| 04 Feb 25 | 0.0 | 0.9 | 0.3 | 0.4 | 0.1 | 1.6 | 0.2 | 0.5 | 0.0 | 4.1 |
| 04 Mar 11 | 0.0 | 0.8 | 0.3 | 0.4 | 0.1 | 1.5 | 0.1 | 0.5 | 0.0 | 3.7 |
| 04 Mar 25 | 0.0 | 0.9 | 0.3 | 0.4 | 0.1 | 1.5 | 0.1 | 0.5 | 0.0 | 3.8 |
| 04 Apr 08 | 0.0 | 0.8 | 0.3 | 0.4 | 0.1 | 1.5 | 0.1 | 0.4 | 0.0 | 3.6 |
| 04 Apr 23 | 0.0 | 0.8 | 0.3 | 0.4 | 0.1 | 1.5 | 0.2 | 0.5 | 0.0 | 3.7 |
| 04 May 20 | 0.0 | 0.8 | 0.3 | 0.4 | 0.1 | 1.5 | 0.2 | 0.5 | 0.0 | 3.8 |

35° TEMPERATURE

| | | | | | | | | | | |
|---|---|---|---|---|---|---|---|---|---|---|
| 04 Jan 26 | 0.0 | 0.9 | 0.3 | 0.4 | 0.1 | 1.5 | 0.1 | 0.5 | 0.0 | 3.8 |
| 04 Feb 12 | 0.0 | 0.8 | 0.3 | 0.4 | 0.1 | 1.5 | 0.1 | 0.5 | 0.0 | 3.7 |
| 04 Feb 25 | 0.0 | 0.8 | 0.3 | 0.4 | 0.1 | 1.5 | 0.1 | 0.5 | 0.0 | 3.7 |
| 04 Mar 11 | 0.0 | 0.8 | 0.2 | 0.3 | 0.1 | 1.4 | 0.1 | 0.4 | 0.0 | 3.5 |
| 04 Mar 25 | 0.0 | 0.6 | 0.2 | 0.2 | 0.1 | 1.0 | 0.1 | 0.3 | 0.0 | 2.5 |
| 04 Apr 08 | 0.0 | 0.8 | 0.2 | 0.3 | 0.1 | 1.4 | 0.1 | 0.4 | 0.0 | 3.3 |
| 04 Apr 23 | 0.0 | 0.8 | 0.2 | 0.3 | 0.1 | 1.4 | 0.1 | 0.4 | 0.0 | 3.5 |
| 04 May 20 | 0.0 | 0.8 | 0.3 | 0.4 | 0.1 | 1.5 | 0.2 | 0.5 | 0.0 | 3.7 |

Catechin (Polyphenol) Legend:
EGC: Epigallocatechin
GC: Gallocatechin
EC: Epicatechin
C: Catechin
EGCg: Epigallocatechin gallate
GCg: Gallocatechin gallate
Ecg: Epicatechin gallate
Cg: Catechin gallate

As indicated by the catechin content over time (Table One, Figure One), the concentration of polyphenols in the skin cream does not decline appreciably over the period tested, even at the higher temperatures considered to be an accelerated aging test. This data indicates continued stability over long periods of time for formulations as described by the present invention.

By contrast, the same test performed on several commercially available skin cream formulations indicated that no catechins could be detected when testing product purchased through normal outlets. These skin creams included those under the tradenames of “Green Beaver's Green tea” and “Jason's Tea Time”. These skin creams do not contain an anhydrous formulation or use of a suitable adsorbent binding carrier.

Although the present invention has been described in detail with particular reference to referred embodiments thereof, it should be understood that the invention is capable of other different embodiments, and its details are capable of modifications in various obvious respects. As is readily apparent to those skilled in the art, variations and modifications can be affected while remaining within the spirit and scope of the invention. Accordingly, the foregoing disclosure and description are for illustrative purposes only, and do not in any way limit the invention, which is defined only by the claims.

1. A composition of matter consisting of an adsorbent binding carrier and a polyphenol in an anhydrous mixture, wherein said polyphenol is adsorbed to said binding carrier.

2. The composition according to claim 1, additionally comprising an anhydrous topical cram, gel or ointment, wherein the polyphenol adsorbed to said adsorbent binding carrier is evenly disbursed within the cream, gel or ointment, and wherein said polyphenol will be released on and into the skin when the cream, gel or ointment is applied thereto.

3. The composition according to claim 2, wherein the polyphenol comprises polyphenols derived from tea (Camellia sinensis) or green tea.

4. The composition according to claim 2, wherein the binding carrier is selected from the group consisting of talcs and clays, alginates, algae, agars, gums, gelatins, celluloses, silica, silica gels, simethicone, salicylates, silicates and silicone resins, tragacanths, calcium carbonates and magnesium and zinc oxides.

5. The composition according to claim 4, wherein the binding carrier is silica or a silica gel.

6. The composition according to claim 4, wherein the binding carrier is a salicylate or a silicate.

7. The composition according to claim 4, wherein the binding is a magnesium or zinc oxide.

8. The composition according to claim 2, wherein the anhydrous topical cream, gel or ointment comprises saturated or unsaturated plant oils or waxes.

9. The composition according to claim 8, wherein the oils or waxes are natural plant oils or waxes.

10-17. (canceled)

18. The composition according to claim 9, wherein said natural plant oils or waxes are shea butter, aloe vera, almond oil, olive oil, avocado oil, coconut oil, jojoba oil or avena sativa oil.

19. A method of formulating a composition according to claim 1 comprising the steps of:
a. triturating an adsorbent binding carrier with a polyphenol until uniform; and
b. subsequently adding an anhydrous topical cream, gel or ointment base.

20. A method of formulating a composition according to claim 2 comprising the steps of:
a. triturating an adsorbent binding carrier with a polyphenol until uniform; and
b. subsequently adding an anhydrous topical cream, gel or ointment base.

21. A method of formulating a composition according to claim 3 comprising the steps of:
a. triturating an adsorbent binding carrier with a polyphenol until uniform; and
b. subsequently adding an anhydrous topical cream, gel or ointment base.

22. A method of formulating a composition according to claim 4 comprising the steps of:
a. triturating an adsorbent binding carrier with a polyphenol until uniform; and
b. subsequently adding an anhydrous topical cream, gel or ointment base.

23. A method of formulating a composition according to claim 5 comprising the steps of:
a. triturating an adsorbent binding carrier with a polyphenol until uniform; and
b. subsequently adding an anhydrous topical cream, gel or ointment base.

24. A method of formulating a composition according to claim 6 comprising the steps of:
a. triturating an adsorbent binding carrier with a polyphenol until uniform; and
b. subsequently adding an anhydrous topical cream, gel or ointment base.

25. A method of formulating a composition according to claim 7 comprising the steps of:
a. triturating an adsorbent binding carrier with a polyphenol until uniform; and
b. subsequently adding an anhydrous topical cream, gel or ointment base.

26. A method of formulating a composition according to claim 8 comprising the steps of:
a. triturating an adsorbent binding carrier with a polyphenol until uniform; and
b. subsequently adding an anhydrous topical cream, gel or ointment base.

27. A method of formulating a composition according to claim 9 comprising the steps of:
a. triturating an adsorbent binding carrier with a polyphenol until uniform; and
b. subsequently adding an anhydrous topical cream, gel or ointment base.

28. A method of formulating a composition according to claim 18 comprising the steps of:
a. triturating an adsorbent binding carrier with a polyphenol until uniform; and
b. subsequently adding an anhydrous topical cream, gel or ointment base.
